(19) United States
(12) Patent Application Publication
(10) Pub. No.: US 2015/0164752 A1
(43) Pub. Date: Jun. 18, 2015

(54) Hair Dye Composition and Method for coloring Hair Using Same

(71) Applicants: LouAnn C. Vena, Scotch Plains, NJ (US); Anjali Abhimanya Patil, Westfield, NJ (US)

(72) Inventors: LouAnn C. Vena, Scotch Plains, NJ (US); Anjali Abhimanya Patil, Westfield, NJ (US)

(73) Assignee: Revlon Consumer Products Corporation, New York, NY (US)

(21) Appl. No.: 14/107,509

(22) Filed: Dec. 16, 2013

(51) Int. Cl. A61K 8/04; A61Q 5/10

(52) U.S. Cl. CPC A61K 8/046; A61Q 5/10

(57) ABSTRACT

The present invention is directed to a dye composition. The composition includes a coloring agent, an oxidizing agent, and a perfluoro-compound. The present invention is also directed to a method for coloring hair using the dye composition. The method includes combining the coloring agent, the oxidizing agent, and the perfluoro-compound to form a coloring composition; applying the coloring composition to at least one hair and allowing foam to develop on the hair; and removing the developed foam from the hair after a period of time.

This application claims priority from copending PCT application PCT/US12/42984, filed Jun. 18, 2012, which claims priority to U.S. provisional application 61/498,686, filed Jun. 20, 2011, now expired, the entire disclosure of which is hereby incorporated by reference.

Hair dye compositions in a liquid or cream form have been in widespread use. However, it is generally difficult to evenly apply those compositions to the head hair. In particular, consumers must adapt to a method for self-application of the compositions evenly to the root portions of the head hair or to the hair in the occipital region. Many products propose to simplify the hair dyeing process by discharging the hair coloring product as a foam, for example, a two-part aerosol type and a one-part non-aerosol type are known. Foam hair dye product that uses a similar combination of ingredients mixed in a shaker type container is also known. Generally, foam hair dye products use surfactants and either a mechanical pump bottle or shake type mixing container to introduce air, to first create a foam which is then dispensed and applied to the hair.

Pre-formed foam is difficult to apply to the root area which is necessary for consumers that color often or for covering up gray hair. The foam, being light and airy, soaks immediately into the first areas it touches, over saturating some areas and under saturating others. The foam often dries out before the coloring process is complete. For these reasons, consumers who use foam color complain of poor uniformity in the hair color, dark ends, dry hair, irritation due to high levels of surfactants, and poor colorfastness and wear.

Accordingly, there is a need to provide a hair dye composition and method for coloring hair using the composition that ensure uniformity, improved wear, and less irritation in a hair dye product suitable for home use.

### SUMMARY OF THE INVENTION

This invention is directed to a dye composition comprising a coloring agent, an oxidizing agent, and a perfluoro-compound.

The composition optionally comprises conditioners, thickeners, surfactants, chelating agents, botanicals, a peroxygenated salt bleach composition, polar solvents, pH adjusters, anti-oxidants, fragrances, opacifying agents and preservatives.

The coloring agent in the composition can be composed of at least one of: an oxidative dye, a direct dye, and a mixture thereof.

This invention is also directed to a method for coloring hair comprising:

a. combining a coloring agent, an oxidizing agent, and a perfluoro compound in a container to form a coloring composition;

b. applying the coloring composition, to at least one hair before the composition begins to form a foam, and allowing the foam to develop on the hair; and

c. washing the developed foam from the hair after about 10 to about 40 minutes.

The hair dye composition according to the present invention develops foam once it has been applied to the hair. Because the hair dye composition is not in a foam form when dispensed and applied, such composition is more easily applied to the hair, especially in the root area, and resulting in improved deposit and evenness, and less color fade.

### DETAILED DESCRIPTION OF THE INVENTION

The terms used in this specification generally have their ordinary meanings in the art, within the context of the invention, and in the specific context where each term is used. Certain terms are discussed below, or elsewhere in the specification, to provide additional guidance to the practitioner in describing the compounds, compositions, and methods of the invention and how to make and use them. Moreover, it will be appreciated that the same thing can be said in more than one way. Consequently, alternative language and synonyms may be used for any one or more of the terms discussed herein, nor is any special significance to be placed upon whether or not a term is elaborated or discussed herein. The use of examples anywhere in this specification, including examples of any terms discussed herein, is illustrative only, and in no way limits the scope and meaning of the invention or of any exemplified term. Likewise, the invention is not limited to the examples presented.

As used herein, “about” or “approximately” shall generally mean within 20 percent, preferably within 10 percent.

The present invention is directed to a dye composition comprising a coloring agent, an oxidizing agent, and a perfluoro-compound.

The perfluoro-compound is any compound that has a boiling point of about room temperature to about 100° C. and an evaporation rate of 2-100 times that of de-ionized water when used in the same composition.

Examples of perfluoro-compounds include, but are not limited to, a perfluoroalkane, perfluoroalkane is selected from the group consisting of a pentafluoropropane, an ethyl perfluorobutyl ether, an ethyl perfluoroisobutyl ether, a perfluoro-2-methylpentane, a perfluoromethylcyclopentane, a perfluorohexane, a perfluoroisohexane, a perfluoromethylcyclohexane, a perfluorodimethylcyclohexane, a perfluoroheptane, a 1,1-dichloro-1-fluoroethane, a 1,1,1,3,3,3-hexafluoropropane, methylnonafluobutyl ether, a methylnonafluoroisobutyl ether, a 1,1,2,2-tetrafluoromethyl ether, a nanafluoro-4(trifluoromethyl)-3-pentanone, and a mixture thereof.

In one non-limiting embodiment, the perfluoroalkane is perfluoroisohexane. In another non-limiting embodiment, the perfluoroalkane is perfluoro-2-methylpentane. In yet another non-limiting embodiment, the perfluoroalkane is a mixture of perfluorohexane, perfluorodecalin, and pentafluoropropane.

Commercial perfluoro-compounds containing perfluoroalkane may be used in the present hair dye composition. For example, PhoenoMulse™ CE-2, available from Phoenix Chemical, Inc., Sommervile, N.J., may be used as the perfluoro-compound in the present hair dye composition. PhoenoMulse™ CE-2 is a volatile perfluoroalkane loaded micellar emulsion containing polyhydroxystearic acid, isononyl isononanoate, ethylhexyl isononanoate, sodium cocamidopropyl PG-dimonium chloride phosphate, perfluorohexane, and butylene glycol. It is understood that other commercial perfluoro-compounds may also be used in the present hair dye composition.

Additional perfluoro-compounds include methyl perfluoroisobutyl ether and methyl perfluorobutyl ether (3M Cosmetic Fluid CF-61), perfluoroisohexane (Flutec PC-1 F2 Chemicals), and perfluorohexane and perfluorodecalin and perfluoropropane (Fiflow BB 61 Creations Couleurs).

The perfluoro-compound is generally present in the hair dye composition in an amount from about 2% w/w to about 40% w/w and can be composed of a single perfluoro-compound or a combination of perfluoro-compounds.

In one embodiment, the oxidizing agent comprises hydrogen peroxide. The hydrogen peroxide used in the present hair dye composition may be an aqueous solution of hydrogen peroxide but could be present in other forms such as encapsulated. Preferably it comprises from about 1 to about 99%, preferably about 10 to about 99%, more preferably about 60 to about 97% of water, and about 5 to about 25%, preferably about 6 to about 20%. Developer compositions are generally sold in the form of 10, 20, 25, 30, or 40 volumes oxidizing agent. The 20 volume hydrogen peroxide developer composition comprises 6% by weight of hydrogen peroxide. The 25 volume hydrogen peroxide developer composition contains about 7.5% of hydrogen peroxide and the 30 volume hydrogen peroxide developer composition about 9%, and the 40 volume developer about 12% hydrogen peroxide, with all weight percentages by weight of the total composition of hydrogen peroxide.

Preferably, the oxidizing agent composition comprises from about 5 to about 50, preferably from about 10 to 40 volume hydrogen peroxide.

The oxidizing agent in the present invention may include other oxidizing agents known in the art. Water-soluble oxidizing agents such as inorganic peroxygen materials capable of yielding hydrogen peroxide in an aqueous solution may be used. Water-soluble peroxygen oxidizing agents are well known in the art and include inorganic alkali metal peroxides such as sodium periodate and sodium peroxide and organic peroxides such as urea peroxide, melamine peroxide, and inorganic perhydrate salt bleaching compounds, such as the alkali metal salts of perborates, percarbonates, perphosphates, persilicates, persulphates and the like. These inorganic perhydrate salts may be incorporated as monohydrates, tetrahydrates etc. Alkyl and aryl peroxides and/or peroxidases may also be used. Mixtures of two or more such oxidizing agents can be used if desired. The oxidizing agents may be provided in aqueous solution or as a powder which is dissolved prior to use. In addition, alkali metal bromates, peracids, and oxidase enzymes, such as peroxidases, 2-electron oxidoreductases, such as uricases, and 4-electron oxygenases, such as laccases, may also be used. Any oxidizing agent used in liquid, cream, or foaming hair color can be used with the perfluoro-compounds of this invention without limitations. Oxidizing agents may be used alone or as a combination of various oxidizing agents in concentrations described above for hydrogen peroxide.

The hair coloring agent used in the present hair dye composition may include any hair coloring agent known in the art. Examples of the hair coloring agent include, but not limited to, oxidative dyes and direct dyes. In one embodiment, the hair coloring agent comprises at least one of an oxidative dye, a direct dye, and a mixture thereof.

Oxidative Dyes

The oxidative dye comprises at least one primary intermediate and, optionally, at least one coupler for the formation of an oxidative dye. It is noted that the primary intermediates and optional couplers that are found in the oxidative dye are generally selected to impart the desired color to the hair.

Primary Intermediates

Suitable ranges of primary intermediates are about 0.0001 to about 6%, preferably about 0.0005 to about 8%, more preferably about 0.001 to about 8% by weight of the hair dye composition.

Primary intermediates are well known for use in hair color, and include ortho or para substituted aminophenols or phenylenediamines, including para-phenylenediamines of the formula:

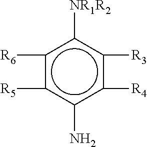

wherein R₁ and R₂ are each independently hydrogen, C₁₋₆ alkyl, or C₁₋₆ alkyl substituted with one or more hydroxy, methoxy, methylsulphonylamino, aminocarbonyl, furfuryl, unsubstituted phenyl, or amino substituted phenyl groups; R₃, R₄, R₅, and R₆ are each independently hydrogen, C₁₋₆ alkyl, C₁₋₆ alkoxy, halogen, or C₁₋₅ alkyl substituted with one or more hydroxy or amino groups.

Specific examples of suitable primary intermediates include, but not limited to, para-phenylenediamine, 2-methyl-1,4-diaminobenzene, 2,6-dimethyl-1,4-diaminobenzene, 2,5-dimethyl-1,4-diaminobenzene, 2,3-dimethyl-1,4-diaminobenzene, 2-chloro-1,4-diaminobenzene, 2-methoxy-1,4-diaminobenzene, 1-phenylamino-4-aminobenzene, 1-dimethylamino-4-aminobenzene, 1-diethylamino-4-aminobenzene, 1-bis(beta-hydroxyethyl)amino-4-aminobenzene, 1-methoxyethylamino-4-aminobenzene, 2-hydroxymethyl-1,4-diaminobenzene, 2-hydroxyethyl-1,4-diaminobenzene, 2-isopropyl-1,4-diaminobenzene, 1-hydroxypropylamino-4-aminobenzene, 2,6-dimethyl-3-methoxy-1,4-diaminobenzene, 1-amino-4-hydroxybenzene, and derivatives and acid or basic salts thereof.

Preferred primary intermediates are p-phenylenediamine, p-aminophenol, o-aminophenol, N,N-bis(2-hydroxyethyl)-p-phenylenediamine, 2,5-diaminotoluene, 1-hydroxyethyl-4,5-diaminopyrazole, 4-amino-3-methylphenol, 6-amino-3-methylphenol, 2-(2-hydroxyethyl)-p-phenyldiamine, tetra-aminopyridine, their salts and mixtures thereof.

Couplers

The oxidative dye may optionally comprise from about 0.0001 to about 10%, more preferably about 0.0005 to about 10%, most preferably about 0.001 to about 8% by weight of the primary intermediate of one or more color couplers. Suitable color couplers include, for example, those having the general formula:

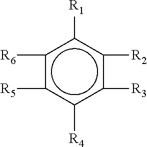

wherein R₁ is unsubstituted hydroxy or amino, or hydroxy or amino substituted with one or more C₁₋₆ hydroxyalkyl groups, R₃ and R₅ are each independently hydrogen, hydroxy, amino, or amino substituted with C₁₋₆ alkyl, C₁₋₆ alkoxy, or C₁₋₆ hydroxyalkyl group; and R₂, R₄, and R₆ are each independently hydrogen, C₁₋₆ alkoxy, C₁₋₆ hydroxyalkyl, or C₁₋₆ alkyl, or R₃ and R₄ together may form a methylenedioxy or ethylenedioxy group. Examples of such compounds include meta-derivatives such as phenols, catechol, meta-aminophenols, meta-phenylenediamines, and the like, which may be unsubstituted, or substituted on the amino group or benzene ring with alkyl, hydroxyalkyl, alkylamino groups, and the like.

Suitable couplers include m-aminophenol, 2,4-diaminotoluene, 4-amino-2-hydroxytoluene, phenyl methyl pyrazolone, 3,4-methylenedioxyphenol, 3,4-methylenedioxy-1-[(beta-hydroxyethyl)amino]benzene, 1-methoxy-2-amino-4-[(beta-hydroxyethyl)amino]benzene, 1-hydroxy-3-(dimethylamino)benzene, 6-methyl-1-hydroxy-3[(beta-hydroxyethyl)amino]benzene, 2,4-dichloro-1-hydroxy-3-aminobenzene, 1-hydroxy-3-(diethylamino)benzene, 1-hydroxy-2-methyl-3-aminobenzene, 2-chloro-6-methyl-1-hydroxy-3-aminobenzene, 1,3-diaminobenzene, 6-methoxy-1,3-diaminobenzene, 6-hydroxyethoxy-1,3-diaminobenzene, 6-methoxy-5-ethyl-1,3-diaminobenzene, 6-ethoxy-1,3-diaminobenzene, 1-bis(beta-hydroxyethyl)amino-3-aminobenzene, 2-methyl-1,3-diaminobenzene, 6-methoxy-1-amino-3-[(beta-hydroxyethyl)amino]-benzene, 6-(beta-aminoethoxy)-1,3-diaminobenzene, 6-(beta-hydroxyethoxy)-1-amino-3-(methylamino)benzene, 6-carboxymethoxy-1,3-diaminobenzene, 6-ethoxy-1-bis(beta-hydroxyethyl)amino-3-aminobenzene, 6-hydroxyethyl-1,3-diaminobenzene, 1-hydroxy-2-isopropyl-5-methylbenzene, 1,3-dihydroxybenzene, 2-chloro-1,3-dihydroxybenzene, 2-methyl-1,3-dihydroxybenzene, 4-chloro-1,3-dihydroxybenzene, 5,6-dichloro-2-methyl-1,3-dihydroxybenzene, 1-hydroxy-3-amino-benzene, 1-hydroxy-3-(carbamoylmethylamino)benzene, 6-hydroxybenzomorpholine, 4-methyl-2,6-dihydroxypyridine, 2,6-dihydroxypyridine, 2,6-diaminopyridine, 6-aminobenzomorpholine, 1-phenyl-3-methyl-5-pyrazolone, 1-hydroxynaphthalene, 1,7-dihydroxynaphthalene, 1,5-dihydroxynaphthalene, 5-amino-2-methyl phenol, 4-hydroxyindole, 4-hydroxyindoline, 6-hydroxyindole, 6-hydroxyindoline, 2,4-diamionphenoxyethanol, and mixtures thereof.

Preferred couplers include, but not limited to, resorcinol, 1-naphthol, 2-methylresorcinol, 4-amino-2-hydroxy toluene, m-aminophenol, 2,4-diaminophenoxyethanol, phenyl methyl pyrazolone, 2-methyl-5-hydroxyethylaminophenol, m-phenylene diamine, 5-(2-hydroxyethylamino)-2-methylphenol, their salts, and mixtures thereof.

Direct Dyes

The hair coloring agent used in the present hair dye composition may include a direct dye which may be used alone or in combination with the above-described oxidative dye. Suitable direct dyes include azo or anthraquinone dyes, nitro dyes (including nitro derivatives of the benzene series), disperse dyes, basic dyes, acid dyes, melanin precursors and mixtures thereof.

Examples of nitro dyes include 2-nitro-paraphenylenediamine, 2-amino-6-chloro-4-nitrophenol, 3-nitro-parahydroxyethylaminophenol, 4-nitro-orthophenylenediamine, 4-amino-3-nitrophenol, 4-hydroxypropylamino-3-nitrophenol, HC Blue No. 2, HC Orange No. 1, HC Red No. 1, HC Yellow No. 2, HC Yellow No. 4, HC Yellow No. 5, HC Red No. 3, N,N-bis-(2-hydroxyethyl)-2-nitro-paraphenylenediamine, indamines, indoaniline, and indophenols.

Examples of disperse dyes include Disperse Violet No. 1, Disperse Blue No. 1, and Disperse Black No. 9; those of the basic dye include Basic Blue No. 99, Basic Brown No. 16, Basic Brown No. 17, Basic Red No. 76, Basic Red No. 51, Basic Yellow No. 57, Basic Yellow No. 87 and Basic Orange No. 31; and those of the acid dye include Orange No. 205 and Red No. 106.

These direct dyes may be used in combination of two or more thereof or the direct dye may be used in combination with the oxidative dye. Suitable amount of the direct dye(s) in the hair dye composition is from 0.001% w/w to about 5% w/w, preferably from about 0.01% w/w to about 3% w/w.

The hair dye composition of the present invention may include a variety of other ingredients that enhance the aesthetic properties and contribute to more efficient coloring of hair. Examples of the other ingredients include, but not limited to, conditioners, thickeners, surfactants, chelating agents, botanicals, a peroxygenated salt bleach composition, polar solvents, pH adjusters, anti-oxidants, fragrances, opacifying agents and preservatives.

Conditioners

The hair dye composition may contain one or more conditioners that exert a conditioning effect on the hair. If present, such conditioners may range from about 0.1 to about 30%, preferably from about 0.5 to about 25%, more preferably from about 1 to about 20% by weight of the hair dye composition of one or more conditioners. Additional example of conditioners are found in U.S. Pat. No. 7,204,861 cols. 23-28 and U.S. Pat. No. 7,566,348 cols. 3-6, both of which are hereby incorporated by reference. Examples of suitable conditioning ingredients include, but are not limited to those set forth: polyquaternium-10, polyquaternium-28, cetrimonium chloride, proteins, and amino acids.

Thickeners

The hair dye composition may contain one or more thickeners that assist in maintaining an increased viscosity of the composition resulting from mixture of the hair coloring agent, the oxidizing agent, and the foaming agent. The amount of thickening agent, if present is about 0.001 to about 10%, preferably about 0.005 to about 4%, more preferably about 0.05 to about 5% by weight of the hair dye composition.

A variety of thickening agents are suitable, including those mentioned above with respect to the oxidative dye composition, in addition to low melting point waxes, carboxyvinyl polymers, and the like. Also suitable are a variety of water soluble anionic thickening polymers such as those disclosed in U.S. Pat. No. 4,240,450, which is hereby incorporated by reference in its entirety. Suggested ranges of such polymers are about 0.01-10%, by weight of the hair dye composition. Examples of such anionic polymers are copolymers of vinyl acetate and crotonic acid, graft copolymers of vinyl esters or acrylic or methacrylic acid esters, cross-linked graft copolymers resulting from the polymerization of at least one monomer of the ionic type, at least one monomer of the nonionic type, polyethylene glycol, and a crosslinking agent, and the like. Preferred thickeners are acrylate copolymers such as steareth-10 allyl ether acrylate copolymer.

Other thickeners, such as those disclosed in U.S. Pat. No. 7,204,861, cols 17-22, which is hereby incorporated by reference, may be suitable for use in the hair dye composition of the present invention.

Surfactants

The hair dye composition may comprise one or more surfactants that may assist in maintaining the composition in the emulsion form if it is an emulsion, or aid in the foaming capability of the composition. Suitable surfactants include anionic surfactants, nonionic surfactants, amphoteric surfactants, and the like. If present, surfactants may range from about 0.001 to about 50%, preferably about 0.005 to about 45%, more preferably about 0.1 to about 40% by weight of the hair dye composition.

Nonionic Surfactants

Suggested ranges of nonionic surfactant, if present, are about 0.01 to about 20%, preferably about 0.05 to about 18%, by weight of the hair dye composition. Suitable nonionic surfactants include alkoxylated alcohols or ethers, alkoxylated carboxylic acids, sorbitan derivatives, alkyl polyglucoside (e.g., decyl glucoside), glyceryl ethers, and the like.

Suitable alkoxylated alcohols, or ethers, are formed by the reaction of an alcohol with an alkylene oxide, usually ethylene or propylene ethylene oxide. Preferably, the alcohol is a fatty alcohol having 6 to 30 carbon atoms, and a straight or branched, saturated or unsaturated carbon chain. Examples of such ingredients include steareth 2-30, which is formed by the reaction of stearyl alcohol and ethylene oxide where the number of repeating ethylene oxide units is 2 to 30; Oleth 2-30, which is formed by the reaction of oleyl alcohol and ethylene oxide where the number of repeating ethylene oxide units is 2 to 30; Ceteareth 2-100, formed by the reaction of a mixture of cetyl and stearyl alcohol with ethylene oxide, where the number of repeating ethylene oxide units in the molecule is 2 to 100; Ceteth 1-45, which is formed by the reaction of cetyl alcohol and ethylene oxide, and the number of repeating ethylene oxide units is 1 to 45; and so on.

Also suitable as the nonionic surfactant are alkyoxylated carboxylic acids, which are formed by the reaction of a carboxylic acid with an alkylene oxide or with a polymeric ether. The resulting products have the general formula:

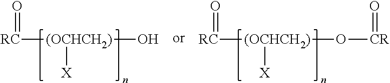

wherein RCO is the carboxylic ester radical, X is hydrogen or lower alkyl, and n is the number of polymerized alkoxy groups. In the case of the diesters, the two RCO groups do not need to be identical. Preferably, R is a C₆₋₃₀ straight or branched chain, saturated or unsaturated alkyl, and n is from 1-100.

Also suitable are various types of alkoxylated sorbitan and alkoxylated sorbitan derivatives. For example, alkoxylation, in particular, ethoxylation, of sorbitan provides polyalkoxylated sorbitan derivatives. Esterification of polyalkoxylated sorbitan provides sorbitan esters such as the polysorbates. Examples of such ingredients include Polysorbates 20-85, sorbitan oleate, sorbitan palmitate, sorbitan sesquiisostearate, sorbitan stearate, and so on.

Anionic Surfactants

Surfactants suitable for use are exemplified in EP 2204160 and EP 2204157, hereby incorporated by reference

If desired the hair dye composition may contain one or more anionic surfactants. Preferred ranges of anionic surfactant are about 0.01 to about 25%, preferably about 0.5 to about 20%, more preferably about 1 to about 15% by weight of the hair dye composition. Suitable anionic surfactants include alkyl and alkyl ether sulfates generally having the formula ROSO₃M and RO(C₂H₄O)ₓSO₃M wherein R is alkyl or alkenyl of from about 10 to 20 carbon atoms, x is 1 to about 10 and M is a water soluble cation such as ammonium, sodium, potassium, or triethanolamine cation.

Another type of anionic surfactant which may be used in the hair dye composition of the present invention are water soluble salts of organic, sulfuric acid reaction products of the general formula:

R₁—SO₃-M

wherein R₁ is chosen from the group consisting of a straight or branched chain, saturated aliphatic hydrocarbon radical having from about 8 to about 24 carbon atoms, preferably 12 to about 18 carbon atoms; and M is a cation. Examples of such anionic surfactants are salts of organic sulfuric acid reaction products of hydrocarbons such as n-paraffins having 8 to 24 carbon atoms, a-olefins, and a sulfonating agent, such as sulfur trioxide.

Also suitable as anionic surfactants are reaction products of fatty acids esterified with isethionic acid and neutralized with sodium hydroxide, or fatty acids reacted with alkanolamines or ammonium hydroxides. The fatty acids may be derived from coconut oil, for example. Examples of fatty acids also include lauric acid, stearic acid, oleic acid, palmitic acid, and so on.

In addition, succinates and succinimates are suitable anionic surfactants. This class includes compounds such as disodium N-octadecylsulfosuccinate; tetrasodium N-(1,2-dicarboxyethyl)-N-octadecylsulfosuccinate; and esters of sodium sulfosuccinic acid, e.g., the dihexyl ester of sodium sulfosuccinic acid, the dioctyl ester of sodium sulfosuccinic acid, and the like.

Other suitable anionic surfactants include olefin sultanates having about 12 to 24 carbon atoms. The term “olefin sultanate” means a compound that can be produced by sulfonation of an alpha olefin by means of uncomplexed sulfur trioxide, followed by neutralization of the acid reaction mixture in conditions such that any sultones, which have been formed in the reaction are hydrolyzed to give the corresponding hydroxy-alkanesulfonates. The alpha-olefin from which the olefin sultanate is derived is a mono-olefin having about 12 to 24 carbon atoms, preferably about 14 to 16 carbon atoms.

Other classes of suitable anionic organic surfactants are the beta-alkoxy alkane sulfonates or water soluble soaps thereof such as the salts of C₁₀₋₂₀ fatty acids, for example coconut and tallow based soaps. Preferred salts are ammonium, potassium, and sodium salts.

Still another class of anionic surfactants include N-acyl amino acid surfactants and salts thereof (alkali, alkaline earth, and ammonium salts) having the formula:

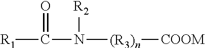

wherein R₁ is a C₈₋₂₄ alkyl or alkenyl radical, preferably C₁₀₋₁₈; R₂ is H, C₁₋₄ alkyl, phenyl, or —CH₂COOM; R₃ is CX₂— or C₁₋₂ alkoxy, wherein each X independently is H or a C₁₋₆ alkyl or alkylester, n is from 1 to 4, and M is H or a salt forming cation as described above. Examples of such surfactants are the N-acyl sarcosinates, including lauroyl sarcosinate, myristoyl sarcosinate, cocoyl sarcosinate, and oleoyl sarcosinate, preferably in sodium or potassium forms; polyglyceryl alkyl ether acetate salt; and ether acetate salt.

Cationic, Zwitterionic or Betaine Surfactants

Certain types of amphoteric, zwitterionic, or cationic surfactants may also be used as the amphiphilic surface active material. Descriptions of such surfactants are set forth in U.S. Pat. No. 5,843,193, which is hereby incorporated by reference in its entirety. If present, suitable ranges of the cationic, zwitterionic or betaine surfactants are about 0.1-15 preferably about 0.5-8 by weight of the hair dye composition.

Amphoteric surfactants that can be used in the compositions of the invention are generally described as derivatives of aliphatic secondary or tertiary amines wherein one aliphatic radical is a straight or branched chain alkyl of 8 to 18 carbon atoms and the other aliphatic radical contains an anionic group such as carboxy, sulfonate, sulfate, phosphate, or phosphonate.

Suitable amphoteric surfactants may be imidazolinium compounds having the general formula:

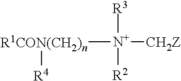

wherein R₁ is C₈₋₂₂ alkyl or alkenyl, preferably C₁₂₋₁₆, cocamidopropyl betaine; R₂ is hydrogen or CH₂CO₂M, R₃ is CH₂CH₂OH or CH₂CH₂OCH₂CHCOOM, R₄ is hydrogen, CH₂CH₂OH, or CH₂CH₂OCH₂CH₂COOM, Z is CO₂M or CH₂CO₂M, n is 2 or 3, preferably 2, M is hydrogen or a cation such as an alkali metal, alkaline earth metal, ammonium, or alkanol ammonium cation. Examples of such materials are marketed under the tradename MIRANOL, by Miranol, Inc.

Also suitable amphoteric surfactants are monocarboxylates or dicarboxylates such as cocoamphocarboxypropionate, cocoamphocarboxypropionic acid, cocoamphocarboxyglycinate, and cocoamphoacetate.

Other types of amphoteric surfactants include aminoalkanoates of the formula:

R—NH(CH₂)ₙCOOM

or iminodialkanoates of the formula:

R—N[(CH₂)ₘCOOM]₂

and mixtures thereof; wherein n and m are 1 to 4, R is C₈₋₂₂ alkyl or alkenyl, and M is hydrogen, alkali metal, alkaline earth metal, ammonium or alkanolammonium. Examples of such amphoteric surfactants include n-alkylaminopropionates and n-alkyliminodipropionates, which are sold under the trade name MIRATAINE by Miranol, Inc. or DERIPHAT by Henkel, for example N-lauryl-beta-amino propionic acid, N-lauryl-beta-imino-dipropionic acid, or mixtures thereof.

Zwitterionic surfactants are also suitable for use in the hair dye compositions of the invention. The general formula for such surfactants is:

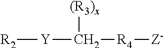

wherein R₂ contains an alkyl, alkenyl, or hydroxy alkyl radical of from about 8 to about 18 carbon atoms, from 0 to about 10 ethylene oxide moieties and 0 or 1 glyceryl moiety; Y is selected from the group consisting of nitrogen, phosphorus, and sulfur atoms; R₃ is an alkyl or monohydroxyalkyl group containing about 1 to 3 carbon atoms; X is 1 when Y is a sulfur atom, and 2 when Y is a nitrogen or phosphorus atom; R₄ is an alkylene or hydroxyalkylene of from about 1 to about 4 carbon atoms, and Z is a radical selected from the group consisting of carboxylate, sulfonate, sulfate, phosphonate, and phosphate groups.

The zwitterionic surfactants also include betaines, for example higher alkyl betaines such as coco dimethyl carboxymethyl betaine, lauryl dimethyl carboxymethyl betaine, lauryl dimethyl alphacarboxyethyl betaine, cetyl dimethyl carboxymethyl betaine, lauryl bis-(2-hydroxyethyl)carboxymethyl betaine, stearyl bis-(2-hydroxypropyl)carboxymethyl betaine, oleyl dimethyl gamma-carboxylethyl betaine, and mixtures thereof. Also suitable are sulfo- and amido-betaines such as coco dimethyl sulfopropyl betaine, stearyl dimethyl sulfopropyl betaine, and the like.

Chelating Agents

The hair dye composition of the present invention may also contain about 0.0001 to about 5%, preferably about 0.0005 to about 3%, more preferably about 0.001 to about 2% by weight of one or more chelating agents which are capable of complexing with and inactivating metallic ions in order to prevent their adverse effects on the stability or effects of the composition. In particular, the chelating agent will chelate the metal ions found in the water and prevent these ions from interfering with the deposition and reaction of the dye with the hair fiber surface. Suitable chelating agents include EDTA and calcium, sodium, or potassium derivatives thereof, HEDTA, sodium citrate, TEA-EDTA, and so on.

Botanicals

The hair dye composition of the present invention may include one or more botanicals. Suitable botanicals include various extracts such as Hypnea musciformis, Gellidiela acerosa, Sargassum filipendula, etc.

Peroxygenated Salt Bleach Composition

The hair dye composition of the present invention may also include, as a lifting composition, a peroxygenated salt bleach composition. The bleach composition may be in the form of a powder, paste, cream, or liquid. The peroxygenated salt composition generally comprises a mixture of peroxygenated salts which are capable of bleaching the hair, particulate fillers, and, if desired, inorganic particulate colorants. An example of the peroxygenated salt composition is a persulfate composition which may be found in the powdered particulate form, or in the form of a cream or paste as described in U.S. Pat. No. 5,888,484 and U.S. Pat. No. 6,613,311, both of which are hereby incorporated by reference in their entirety.

Peroxygenated Salts

The peroxygenated salt composition comprises about 15 to about 65%, preferably about 20 to about 60%, more preferably about 25 to about 55% by weight of the total peroxygenated salt composition of one or more inorganic peroxygenated salt which may be alkali metal or alkaline earth metal persulfates, ammonium persulfate, perborates, or percarbonates.

Particulate Fillers

The peroxygenated salt composition may comprise one or more particulate fillers. Preferably, the peroxygenated salt composition comprises about 5 to about 60%, preferably about 8 to about 55%, more preferably about 10 to about 50% by weight of the total peroxygenated salt composition of the particulate fillers. The term “particulate filler” means a generally inert particulate having a particle size of about 0.1 to about 250 microns. The particulate fillers provide volume and, when mixed with the peroxygenated salt, dilute the peroxygenated salt particles. A variety of particulate fillers are suitable including inorganics, inorganic salts, hydrophilic colloids, carbohydrates, soaps, alkyl sulfates, and the like.

Examples of inorganics include silica, hydrated silica, alumina, attapulgite, bentonite, calcium oxide, chalk, diamond powder, diatomaceous earth, fuller's earth, hectorite, kaolin, mica, magnesium oxide, magnesium peroxide, montmorillonite, pumice, talc, tin oxide, zeolite, zinc oxide, and the like.

Examples of suitable hydrophilic colloids include hydroxyethylcellulose, locust bean gum, maltodextrin, methylcellulose, agar, dextran, dextran sulfate, gelatin, pectin, potassium alginate, sodium carboxymethylchitin, xanthan gum, and the like.

Examples of suitable carbohydrates include sugars such as glucose, sucrose, maltose, xylose, trehelose, and derivatives thereof, in particular sugar esters of long chain, C₁₄₋₃₀ fatty acids, as well as dextrins, cellulosics, and derivatives thereof.

Examples of soaps and alkyl sulfate particles that may act as particulate fillers include the aluminum, sodium, and potassium salts of fatty acids such as aluminum distearate, aluminum isostearate, aluminum myristate, calcium behenate, calcium stearate, calcium behenate, magnesium stearate, magnesium tallowate, potassium palmitate, potassium stearate, potassium oleate, sodium stearate, sodium oleate, sodium myristate, sodium palmitate, and the like. Suitable alkyl sulfates include sodium lauryl sulfate, sodium cetyl sulfate, sodium myristyl sulfate, sodium octyl sulfate, sodium laureth, and the like.

Inorganic Colorants

If desired, the peroxygenated salt composition may comprise about 0.01 to about 2%, preferably about 0.05 to about 1%, more preferably about 0.1 to about 1% by weight of the total peroxygenated salt composition of an inorganic colorant. The inorganic colorant is preferably in the particulate form and will provide a subtle coloration to the powder composition to make it more aesthetically pleasing for commercial purposes. A preferred inorganic colorant for use in the peroxygenated salt composition is ultramarine blue.

Polar Solvents

The hair dye composition may also comprise a variety of polar solvents other than water, including mono-, di-, or polyhydric alcohols, and similar water soluble ingredients. If present, such polar solvents may range from about 0.01 to about 25%, preferably about 0.05 to about 15%, more preferably about 0.1 to about 10% by weight of the hair dye composition. Examples of suitable monohydric alcohols include ethanol, isopropanol, benzyl alcohol, butanol, pentanol, ethoxyethanol, and the like. Examples of dihydric, or polyhydric alcohols, as well as sugars and other types of humectants that may be used include glucose, fructose, mannose, mannitol, malitol, lactitol, inositol, and the like. Suitable glycols include propylene glycol, dipropylene glycol, butylene glycol, ethylene glycol, polyethylene glycols having from 4 to 250 repeating ethylene glycol units, ethoxydiglycol, and the like, Many of these types of alcohols also serve also serve as penetration enhancers, meaning that they enhance penetration of primary intermediates and couplers into the hair shaft by virtue of their tendency to act as humectants and swell the hair shaft.

In one embodiment of the invention, the composition comprises water in addition to one or more polar solvents, which are dihydric alcohols. In such embodiment, the hair dye composition comprises about 0.001 to about 20%, preferably about 0.005 to about 10%, more preferably about 0.001- to about 10% by weight of the non-aqueous polar solvent.

pH Adjusters

The hair dye composition of the present invention may further comprise small amounts of acids or bases to adjust the pH of the composition to the desired pH range of greater than about 7.0 to 12.0. Suitable acids include hydrochloric acid, phosphoric acid, erythorbic acid, and the like. Suitable bases include sodium hydroxide, potassium hydroxide, ammonia, and the like. Also suitable are primary, secondary, or tertiary amines or derivative thereof such as aminomethyl propanol, monoethanolamine, and the like. Suggested ranges of pH adjusters are from about 0.00001 to about 10%, preferably about 0.00005 to about 10%, more preferably about 0.0001 to about 9% by weight of the hair dye composition.

Preservatives

The hair dye composition may also contain one or more preservatives. Suggested ranges are about 0.0001 to about 8%, preferably 0.0005 to about 7%, more preferably about 0.001 to about 5% by weight of the hair dye composition. Suitable preservatives include methyl, ethyl, and propyl paraben, hydantoins, and the like.

Antioxidants

The hair dye composition may further include one or more antioxidants or reducing agents. If present, the antioxidants or reducing agents are present in the composition from about about 0.1 to about 2% by weight of the hair dye composition. Suitable antioxidants or reducing agents include sodium sulfite, bisulfite salts, thioglycolate salts, thiosulfate salts, erythorbic acid, ascorbic acid, and the like.

Various tests may be carried out to compare hair colored using the hair dye composition of the present invention and hair colored using other hair dye compositions. For example, images of hair are analyzed based on color saturation/chroma (using the L*a*b* color scale); hue/tone (also using the L*a*b* color scale); color evenness which involves a pixel-based assessment of banding irregularities typically observed in hair color and a longitudinal assessment since banding/streaking worsens as hair fades over time; and overall grayness (using the L*a*b* color scale, focusing on quantifying discrete areas not colored by product). The principles of hair color analysis are well-established and have been described in Ford et al., “Colour Space Conversions,” Aug. 11, 1998, available at http://www.poynton.com/PDFs/coloureq.pdf.

More specifically, to test the advantages of the hair dye composition of the present invention over other hair dye compositions, the following test may be carried out.

Hair swatches used for testing may be about 1 gm, about 1.5 gm, or about 2 gm. The hair swatches may be yak hair, Asian hair, Caucasian hair, African hair, African-American hair, natural hair, fully or partially (25%, 50%, 75%, or 95%) gray hair. The hair may have different natural colors or bleached.

For each test, a clean glass plate or Lanetta card or any even hard flat surface and a Boston-Bradley adjustable blade applicator may be used. The hair coloring agent and the oxidizing agent are mixed with the foaming agent or water at an amount of about 0.1 gm to about 25 gm. A suitable size puddle (about 25 gm) of the mixture is poured on the glass plate or Lanetta card and drawn down immediately to a uniform film. A hair swatch is gently laid on top of the draw down and a flat bar or plate is placed on top of the swatch for a few seconds and then removed to ensure at least a portion of hair touches the dye. The swatch is allowed to remain for about 25 minutes and then rinsed with water. Hair conditioner is applied for about 2 minutes and the swatch is rinsed off with water and blow dried.

Each of the swatches may be measured by Datacolor ColorTools QC (version 3.0.5) spectrocolorimeter. The chromaticity (c*) of the swatches are measured from values of a*, b*, in the L*a*b* international color notation system. The degree of lightening is determined from the change in L (lightening), a (red), and b (yellow) values. The Datacolor ColorTools manual is hereby incorporated by reference in its entirety.

Each of the swatches may also be measured by SmartProbe 400 which is a portable colorimeter that measures color in the L*a*b* international color notation system. The degree of lightening is determined from the change in L (lightening), a (red), and b (yellow) values. The SmartProbe. 400 manual is hereby incorporated by reference in its entirety. Turn on SmartProbe 400, a portable colorimeter. Remove the protective cover from the probe. Place the probe of the SmartProbe 400 flat on the surface of the calibration tile and click the button on the side of the probe twice. L, a, b will appear on the LED display, instrument is now calibrated and ready for sample measurements. Instrument will not take any readings without calibration, an error will occur.

The hair dye composition may also be tested in a salon in which the composition is applied to hair and the color is evaluated using SmartProbe 400.

Another way to test the hair dye composition is to use Warp Stripe 13 Fiber Fabric, Style #43 from Testfabrics, Inc to evaluate the hair dye composition with and without the foaming agent. In this test, a ½″ wide vertical strip of the fabric is coated with 3 gm of a mixture of hair coloring agent and oxidizing agent, mixed with a foaming agent or water at an amount of 0.1 gm to 25 gm. Each of the mixtures is applied with an applicator tip and is allowed to remain on the fabric for 25 minutes. The strips are held vertically and gently rinsed off with water. Each of the colors of the strips is evaluated by Datacolor ColorTools QC (version 1.2.1) spectrocolometer and/or by SmartProbe 400. The area covered by the color may be determined by either cutting out the colored area and weighing it or by digitally measuring the pixels.

The present invention is also directed to a method for coloring hair using the hair dye composition of the present invention. The method comprises providing a hair coloring agent, an oxidizing agent, and a perfluoro-compound; combining the hair coloring agent, the oxidizing agent, and the perfluoro-compound to form a coloring composition; applying the coloring composition to at least one hair and allowing foam to develop on the hair; and removing the developed foam from the hair after a period of time.

In one embodiment, the coloring composition is applied to a desired amount of hair and the developed foam covers the desired amount of hair. In another embodiment, the foam is left on the hair for between 5 and 30 minutes.

Hair colored according to the method of the present invention exhibits improved uniformity and improved colorfastness and wear.

### Example 1

8 gm standard hair coloring agent (dark brown) was mixed well with about 8 gm 20 volume oxidizing agent and about 2.4 gm foaming agent 1. The control substituted water for foaming agent. On a clean glass plate 65 mil wet film was drawn down using a Boston-Bradley adjustable blade applicator. About 1 gm yak hair tress was gently laid on top of the draw down. A flat bar weighing about 33 gm was placed on top of the tress for 15 seconds and removed. The tress was allowed to remain on the draw down for 25 minutes. Then it was rinsed with water, hair conditioner was applied and the tress was rinsed off with water and the hair was blow dried.

The chromaticity of the tresses was measured for the front and back of the tress and compared using the datacolor ColorTools QC (version 3.0.5) spectrocolorimeter, hereby incorporated by reference in the entirety. The chromaticity (c*) of the tresses was measured from values of a*, b*, in the CIE La*b* color system. The degree of lightening was determined from the change in L (lightening), a (red), and b (yellow) values. Delta L is a measure of uniformity. The results were as follows:

| | L | a | b | ΔL | ΔE |
|---|---|---|---|---|---|
| Medium brown color + | 35.94 | 3.54 | 8.63 | 3.16 | 3.27 |
| oxidizing agent + foaming | | | | | |
| agent 1 Front | | | | | |
| Medium brown color + | 39.1 | 3.16 | 9.38 | | |
| oxidizing agent + foaming | | | | | |
| agent 1 Back | | | | | |
| Medium brown color + | 37.69 | 3.84 | 9.88 | 15.47 | 15.95 |
| oxidizing agent + water | | | | | |
| Front | | | | | |
| Medium brown color + | 53.15 | 1.89 | 13.25 | | |
| oxidizing agent + water | | | | | |
| Back | | | | | |

### Foaming Agent 1: PhoenoMulse CE-2

The above results illustrate that when foaming agent is used color is deposited more uniformly from front to back of tress. This is evidenced by smaller numbers in the ΔL and ΔE columns for foaming agent compared to no foaming agent, which means hair tresses are more uniform in color with foaming agent.

ΔE was calculated based on CIE1976 (attached as pdf)

### Example 2

The hair tresses were colored as described in example 1. The chromaticity of the tresses was measured for the front and back of the tress and compared using the datacolor ColorTools QC (version 3.0.5) spectrocolorimeter.

| | L | a | b | ΔL | ΔE |
|---|---|---|---|---|---|
| Medium brown color + | 35.94 | 3.54 | 8.63 | 1.75 | 2.18 |
| oxidizing agent + foaming | | | | | |
| agent 1 Front | | | | | |
| Medium brown color + | 37.69 | 3.84 | 9.88 | | |
| oxidizing agent + water | | | | | |
| Front | | | | | |
| Medium brown color + | 39.1 | 3.16 | 9.38 | 14.06 | 14.63 |
| oxidizing agent + foaming | | | | | |
| agent 1 Back | | | | | |
| Medium brown color + | 53.15 | 1.89 | 13.25 | | |
| oxidizing agent + water | | | | | |
| Back | | | | | |

### Foaming Agent 1: PhoenoMulse CE-2

The above results illustrate that when foaming agent is used more color is deposited and the hair is darker. Lower the value of L, more intense is the hair color. The L values for the front and back of the hair tress with foaming agent are lower than the corresponding values without foaming agent. This illustrates that more color is deposited on the front and back of the hair with foaming agent.

### Example 3

Warp Stripe 13 Fiber Fabric, Style #43 from Test Fabrics, Inc was used to evaluate hair color with and without foaming agent. A ½″ wide vertical strip of the fabric was coated with 3 gm mixture of hair color and developer from the hair coloring kit, mixed with a foaming agent (PhoenoMulse CE-1) or water (18 gm) The mixture was applied with the applicator tip and was allowed to remain on the fabric for 25 minutes and then was held vertically and gently rinsed off with water and dried. The area covered by the color was determined by cutting out the colored area and weighing it.

| | |
|---|---|
| Weight of the fabric with foaming agent 2 | 0.69 gm |
| Weight of the fabric without foaming agent 2 | 0.51 gm |

The above results illustrate that after the mixture was applied to the fabric, foam expands and covers more area.

### Example 4

Weight loss was determined as follows: About 1 gm yak hair tress was placed in a petridish. About 8 gm of the sample was added on top of the hair tress. Then hair tress was turned from back to front and about 8 gm of the sample was added on top of the hair tress. The amount of sample evaporated with time was recorded for 30 min or till most of the sample was evaporated if less than 30 min. The average weight loss was calculated by dividing the total weight loss by time taken for evaporation. The greater weight loss is linked to the perfluoro-compound and is found to demonstrate the best color results.

| Test sample | Average weight loss gm/min |
|---|---|
| Water | 0.01 |
| Isopropanol | 0.11 |
| Pentane | 1.0 |
| Phoenomulse CE-1 | 0.02 |
| Phoenomulse CE-2 | 0.02 |
| Perfluoroisohexane | 0.86 |
| Methyl perfluoroisobutyl ether and | 0.66 |
| methyl nonafluorobutyl ether | |

### Example 5

Salon evaluation: Salon panelists were recruited for this study. Hair color (including competitors foam hair color) was applied as per instructions on the box. A panelist's head was evaluated by the trained salon evaluators (expert graders) for hair color tone, evenness of color, color intensity, gray coverage, depth of color, amount of banding, and color vibrancy. These attributes were evaluated on the scale of 1 to 5 with 1 representing “poor” results and 5 representing “excellent” result. Evaluation scale: 1=Poor, 2=Fair, 3=Good, 4=Very good, 5=Excellent.

In addition to the evaluations by expert graders, colorimeter measurements were also taken on the panelists head using the SmartProbe 400, a portable colorimeter by IMS Inc. which employs the CIE La*b* color system. A chunk of hair about 2 inches wide was taken; hair was combed flat against a black hard plastic board. Hair was held tightly in place against a board with a comb. Measurements were taken in a straight line at the root area, in the middle and at the end of the hair strand. Measurements were taken in the front, middle, and back of the head. Measurements were obtained for both sides of the head, in the front, middle and the back. Multiple measurements were taken and the average is used as the result, Standard deviation was calculated. Smaller the standard deviation, more uniform is the color.

The color difference, ΔE was calculated based on CIE 1994. (Attached as scanned document) Calibration of SmartProbe 400, a portable colorimeter by IMS Inc.

What is claimed is:

1. A composition comprising a coloring agent, an oxidizing agent, and a perfluoro-compound.

2. The composition of claim 1 wherein the composition optionally comprises conditioners, thickeners, surfactants, chelating agents, botanicals, a peroxygenated salt bleach composition, polar solvents, pH adjusters, anti-oxidants, fragrances, opacifying agents and preservatives.

3. The composition of claim 2 wherein the composition optionally comprises a surfactant.

4. The composition of claim 1 wherein the perfluoro-compound comprises a perfluoroalkane.

5. The composition of claim 4 wherein the perfluoroalkane is selected from a group consisting of a perfluoroalkane, perfluoroalkane is selected from the group consisting of a pentafluoropropane, an ethyl perfluorobutyl ether, an ethyl perfluoroisobutyl ether, a perfluoro-2-methylpentane, a perfluoromethylcyclopentane, a perfluorohexane, a perfluoroisohexane, a perfluoromethylcyclohexane, a perfluorodimethylcyclohexane, a perfluoroheptane, a 1,1-dichloro-1-fluoroethane, a 1,1,1,3,3,3-hexafluoropropane, methylnonafluobutyl ether, a methylnonafluoroisobutyl ether, a 1,1,2,2-tetrafluoromethyl ether, a nanafluoro-4(trifluoromethyl)-3-pentanone, and a mixture thereof.

6. The composition of claim 4 wherein the perfluoroalkane is perfluoroisohexane.

7. The composition of claim 4 wherein the perfluoroalkane is perfluoro-2-methylpentane.

8. The composition of claim 4 wherein the perfluoroalkane is a mixture of the perfluorohexane, perfluorodecalin, and pentafluoropropane.

9. The composition of claim 1 wherein the perfluoro-compound is present in the composition in an amount from about 2% w/w to about 40% w/w.

10. The composition of claim 1 wherein the oxidizing agent comprises hydrogen peroxide.

11. The composition of claim 1 wherein the oxidizing agent is present in the composition in an amount from about 10-40 volumes.

12. The composition of claim 1 wherein the coloring agent comprises at least one of an oxidative dye, a direct dye, and a mixture thereof.

13. The composition of claim 12 wherein the coloring agent comprises an oxidative dye in an amount from about 0.0001 to about 8% by weight of the composition.

14. The composition of claim 12 wherein the coloring agent comprises a direct dye in an amount from about 0.001 to about 8% by weight of the composition.

15. A method for coloring a hair comprising:
a. combining a coloring agent, an oxidizing agent, and a perfluoro compound in a container to form a coloring composition;
b. applying the coloring composition, to at least one hair before the composition begins to form a foam, and allowing the foam to develop on the hair; and
c. washing the foam from the hair after about 10 to about 40 minutes.

16. The method of claim 15 wherein the coloring composition optionally comprises conditioners, thickeners, surfactants, chelating agents, botanicals, a peroxygenated salt bleach composition, polar solvents, pH adjusters, anti-oxidants, fragrances, opacifying agents and preservatives combined in (14 a).

17. The method of claim 15 wherein a surfactant is optionally combined in (14 a).

18. The method of claim 15 wherein the foam is left on the hair for about 25 minutes.

19. The method of claim 13 wherein the oxidizing agent comprises hydrogen peroxide.

20. The method of claim 13 wherein the coloring agent comprises at least one of: an oxidative dye, a direct dye, and a mixture thereof.
